(19) United States
(12) Patent Application Publication
(10) Pub. No.: US 2012/0198715 A1
(43) Pub. Date: Aug. 9, 2012

(54) APPARATUS AND METHOD FOR MEASURING A BODY PART

(75) Inventor: Jason Paul Eaton, Hunker, PA (US)

(73) Assignee: KONINKLIJKE PHILIPS ELECTRONICS N.V., EINDHOVEN (NL)

(21) Appl. No.: 13/501,314

(22) PCT Filed: Sep. 14, 2010
(86) PCT No.: PCT/IB2010/054142

§ 371 Date: Apr. 11, 2012

Related U.S. Application Data
(60) Provisional application No. 61/251,752, filed on Oct. 15, 2009.

(51) Int. Cl. A61B 5/103

(52) U.S. Cl. 33/512

(57) ABSTRACT

An apparatus for measuring a characteristic of a body part, such as an infant's head, includes a flexible substrate, a plurality of sensing elements provided along the substrate, and an electronic system. Each of the sensing elements (i) has a component wherein an electrical characteristic of the component changes predictably in response to the component being bent, and (ii) provides a signal that is indicative of the current value of the electrical characteristic. The electronic system is structured to receive the signal of each sensing element and determine a measure of a degree of curvature of the sensing element based on the received signal. The electronic system is also structured to determine, based one or more of the measures, a representation of a curvature of a segment, such as a loop, defined by a selected one or more of the sensing elements.

The present invention relates to the measurement of characteristics relating to body parts, such as a human head (e.g., an infant's head), and more particularly, to an apparatus and method for measuring a characteristic, such as the degree of curvature or surface profile, of a body part or portion of a body part, such as a human head, that employs a plurality of sensing elements having electrical characteristics that vary in a predictable manner as the sensing elements are bent.

Babies are born with relatively compliant skull structures, and the more prematurely that a baby is born, the more compliant the skull structure. When an infant is placed in one position for a long period of time, the effect of gravity on the infant's head can be sufficient to cause semi-permanent deformation of the skull structure. The fact that premature infants are naturally relatively immobile, and the fact that many care interventions render them even more immobile, can create even higher incidences of head deformation, known as plagiocephaly, among neonates. Even babies which are born full-term may experience head deformation over time, especially since parents are advised to place babies on their backs to sleep as a preventative measure against Sudden Infant Death Syndrome (SIDS).

One of the difficulties with plagiocephaly is that it is quite difficult to detect until damage becomes visually noticeable, at which point reversing the damage is more difficult. Clinicians currently use measuring tapes to determine head circumference of infants, which is commonly used as an index of growth. This measurement is also often compared with other physiological measurements for indicators of conditions which may cause disproportionate size of the head relative to other parts of the body.

There are no devices currently available that enable the shape of the head, and in particular the curvature of the head, to be simply and accurately monitored over time so that adaptations in care to help reduce and/or eliminate plagiocephaly, such as repositioning the infant or using various products designed for cushioning or positioning the head, can be employed before the damage becomes visually noticeable and/or permanent. Current devices also do not enable the shape of the head to be measured without requiring significant manipulation of the position of the head, which places considerable stress on premature infants.

In one embodiment, an apparatus for measuring a characteristic of a body part is provided that includes an elongated, flexible, inextensible substrate, a plurality of sensing elements provided along the substrate, and an electronic system, each of the sensing elements being operatively coupled to the electronic system. Each of the sensing elements (i) has a component wherein an electrical characteristic of the component (such as resistance, inductance or capacitance) changes predictably in response to the component being bent, and (ii) provides a signal that is indicative of a current value of the electrical characteristic. The electronic system is structured to receive the signal of each of the sensing elements and determine a measure of a degree of curvature (such as radius of curvature) of the sensing element based on the received signal associated with the sensing element. The electronic system is also preferably structured to determine, based on the measure associated with each of a selected one or more of the sensing elements, a representation of a curvature of a segment defined by the selected one or more of the sensing elements. The electronic system may also be structured to determine curvilinear distance of the segment. In the preferred embodiment, the substrate, the sensing elements and the electronic system, are provided together in a portable device.

The segment may be a closed loop, and the electronic system may also be structured to determine that the closed loop has been formed, and perform a calibration adjustment for each of the second plurality of the sensing elements. The segment may also be something other than a closed loop, such as curved portion of the device.

In one particular embodiment, the apparatus includes a mechanism for determining that the closed loop has been formed and for identifying the sensing elements included in the closed loop. The mechanism may include a plurality of conductive pads provided along the substrate and a conductive element provided at a distal end of the substrate. In another particular embodiment, the apparatus may include a mechanism for identifying the sensing elements of the segment that includes a plurality of conductive pads provided along the substrate and a conductive element that is slideable along the substrate.

The electronic system may also be structured to determine an index of curvature deformity based on the measure associated with each of a selected set sensing elements, wherein the index of curvature deformity is determined by calculating a ratio comparing a smallest one of the measures in the set with a largest one of the measures in the set.

Also provided is a method of measuring a characteristic of a body part that includes providing a plurality of sensing elements, each of the sensing elements (i) having a component wherein an electrical characteristic of the component changes predictably in response to the component being bent, and (ii) providing a signal that is indicative of a current value of the electrical characteristic, wrapping the sensing elements around at least a portion of the body part, receiving the signal of each of the sensing elements when the sensing elements are wrapped around the at least a portion of the body part, and determining a measure of a degree of curvature of each of the sensing elements based on the received signals. The method may also includes determining based on each measure a representation of a curvature of a segment defined by the plurality of the sensing elements when the sensing elements are wrapped around the at least a portion of the body part.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

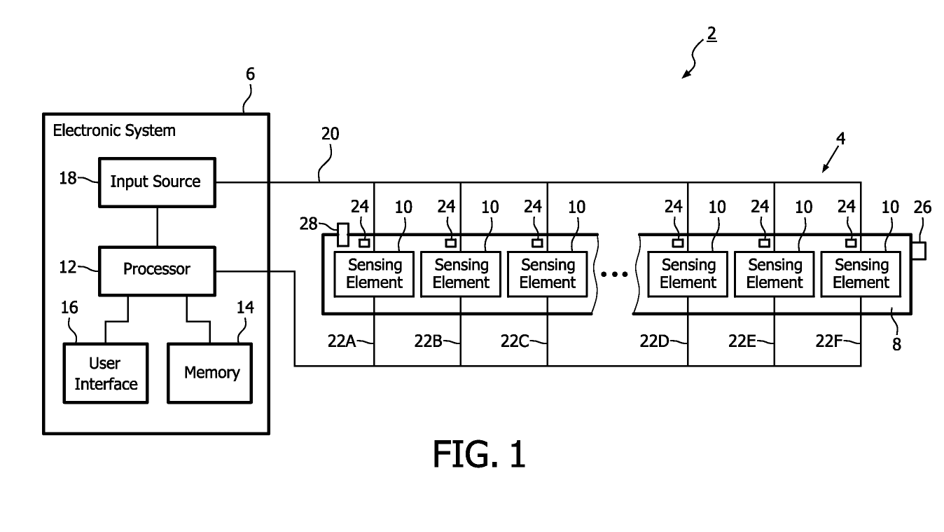

FIG. 1 is a schematic diagram of a measuring apparatus according to one particular embodiment of the present invention.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed, herein, the statement that two or more parts or components are “coupled” together shall mean that the parts are joined or operate together either directly or through one or more intermediate parts or components.

As employed herein, the statement that two or more parts or components “engage” one another shall mean that the parts exert a force against one another either directly or through one or more intermediate parts or components.

As employed herein, the term “number” shall mean one or an integer greater than one (i.e., a plurality).

FIG. 1 is a schematic diagram of measuring apparatus 2 according to one particular embodiment of the present invention. Measuring apparatus 2 includes sensor strip 4 operatively coupled to electronic system 6. Sensor strip 4 includes elongated substrate 8 preferably made of a flexible, bendable, inelastic, inextensible material such as, without limitation, a flexible metal or plastic film. A plurality of sensing elements 10 are provided and positioned (via an appropriate attachment mechanism such as an adhesive) end to end along the length of sensor strip 4. Preferably, sensor strip 4 is similar in shape, size and thickness to known measuring tapes.

Each sensing element 10 includes a component or portion wherein an electrical characteristic of that component or portion, such as, without limitation, resistance, inductance, or capacitance, changes predictably and consistently in response to the component or portion, and thus the associated sensing element 10, being bent. Accordingly, the different values of the particular electrical characteristic that may result from the sensing element 10 being bent can be measured and then correlated to and or used to calculate particular degrees of deflection or curvature of the sensing element 10. In other words, for each sensing element 10, the current value of the particular electrical characteristic (e.g., resistance, inductance, or capacitance) may be used to determine a measure of the current degree of deflection or curvature (e.g., radius of curvature) of the sensing element 10.

One suitable device that may be used for each sensing element 10 is described in U.S. Pat. No. 7,248,142, the disclosure of which is incorporated herein by reference. In particular, the '142 patent describes a sensing device that includes a layer of variable resistance material that has a finite, base electrical resistance when the layer, and thus the sensing device, is flat (not bent), wherein the electrical resistance of the layer changes in predictable way as the layer, and thus the sensing device, is deflected or bent. In the particular embodiments described in the '142 patent, the greater the amount of deflection, the greater the resistance of the layer of material. As a result, different resistance values that result from the sensing device being bent can be measured and used to determine particular degrees of deflection or curvature of the sensing device. The sensing device of the '142 patent just described is meant to be exemplary only and not limiting, and it will be appreciated that other suitable sensing devices of different designs may also be employed within the scope of the present invention.

Returning to FIG. 1, electronic system 6 is operatively coupled to sensor strip 4, and in particular to each sensing element 10 of sensor strip 4. Electronic system 6 includes processor 12, which may be, for example and without limitation, a microprocessor, a microcontroller, or some other suitable custom designed controller circuit (e.g., an application specific integrated circuit (ASIC)). Memory 14 is coupled to processor 12. Memory 14 can be any of a variety of types of internal and/or external storage media such as, without limitation, RAM, ROM, EPROM(s), EEPROM(s), and the like, that provides a storage medium for data and software executable by processor 12 for controlling the operation of measuring apparatus 2 as described herein. Also, processor 12 and memory 14, while shown as separate blocks in FIG. 1, may be combined into a single component or device (i.e., with memory 14 being internal to processor 12). Electronic system 6 further includes user interface 16 operatively coupled to processor 12 for enabling data and/or instructions to be input into and/or output from electronic system 6. In one particular embodiment, user interface 16 includes a keypad or the like for inputting data and control instructions into electronic system 6, and a display, such as an LCD, for displaying information relating to the measurements made by measuring apparatus 2 as described herein.

Electronic system 6 also includes input source 18 for generating electrical input signal 20 that is provided to each sensing element 10. According to an aspect of the present invention, each sensing element 10 will, in response to receipt of electrical input signal 20, generate a specific output signal 22 (labeled 22A-22F in FIG. 1) that is indicative of the current value of the particular varying electrical characteristic (e.g., resistance, inductance, capacitance) of the associated sensing element 10. Output signals 22 are provided to processor 12 of electronic system 6. According to an aspect of the invention, processor 12 is programmed to determine a measure, such as radius of curvature, of the degree of curvature of each sensing element 10 based on the particular received output signal 22 associated with the sensing element 10.

In addition, processor 12 is programmed to determine a representation of the curvature of a segment defined by a particular, selected number of the sensing elements 10 based on the determined measure of degree of curvature associated with each sensing element 10 in the segment. That segment may include a particular, selected number of the sensing elements 10 that are formed into a closed loop, or a particular, selected number of the sensing elements 10 that form an arc. Thus, in the preferred embodiment, when sensor strip 4 is formed into a closed loop, processor 12 is programmed to determine a representation of the curvature and/or outer contour/shape of the loop based on each measure of degree of curvature of the particular sensing elements 10 included in the loop (see description below of how the particular sensing elements 10 of interest may be identified). Thus, in operation, sensor strip 4 may be wrapped fully or partially around the head of an infant and, based on output signals 22 received from the appropriate ones of the sensing elements 10, a representation of the curvature and/or outer contour/shape of the infant's head (or a portion thereof) may be determined by processor 12. That data may be stored in memory 14, and/or may be output to a clinician computer system for subsequent storage, analysis and/or use.

In addition, processor 12 may be programmed to perform a self calibration when sensor strip 4 is formed into a complete closed loop (for example, as detected as described below), as processor 12 will expect that in such as condition, the individual curvature measures associated with each involved sensor element 10 should together form a closed loop. However, depending on the accuracy of the sensor elements 10, the calculated curve may not form a closed loop. The error of each sensor element 10 is essentially multiplied by the number of elements between that sensor element 10 and the end of the loop. Because these errors are compounded, the combined margin of error of all of these sensor elements 10 could result in a calculated arc that does not form a closed loop. If the processor 12 detects, either through a built-in loop detection means, or an indication by the user, that a closed loop was formed during the measurement, the processor 12 could do a real-time uniform adjustment to sensor calibration in order to close the calculated loop, and thus improve accuracy.

In the preferred embodiment, measuring apparatus 2 is provided with a mechanism for determining which particular ones of the sensing elements 10 are to be used in determining the representation of the curvature of a segment of the sensor strip 4. In other words, a mechanism is provided for defining which particular ones of the sensing elements 10 are included in the segment of interest so that only the measures of curvature associated with those particular sensing elements 10 will be used by the processor 12 to determine the representation of the curvature of the segment (the measures of curvature associated with the other sensing elements 10 will in that instance be ignored). That mechanism may include, in one embodiment, a plurality of conductive pads 24 provided along the length of the substrate 8 and a conductive element 26 provided at a distal end of the substrate 8. In this configuration, a circuit can be completed by causing the conductive element 26 to contact a specific pad 24. Such contact can be used as an indication that a closed loop has been formed. In addition, the identification of the specific pad 24 that is contacted can be used to determine which sensing elements 10 are of interest (i.e., within the closed loop), and which sensing elements 10 are not of interest (i.e., outside the closed loop). Also, the curvilinear distance of the segment (e.g., the circumference of a head), which may also be of interest to caregivers, can be determined from the identification of the specific pad 24 that is contacted (based on a known spacing of the pads and/or a known length of sensing elements 10). In another embodiment, that mechanism may also include a plurality of conductive pads 24 provided along the length of the substrate 8 and slideable conductive element 28 that is movable along the length of the substrate 8 such that a circuit is completed each time slideable conductive element 28 contacts a pad 24. Based on the circuit that is completed, the processor 12 can determine which pad 24 is being contacted and thus the length of the sensor strip 4 that is of interest (and therefore the particular sensing elements 10 that are of interest.) Also, the determined length can be used by processor 12 in order to determine the curvilinear distance of the segment, which, as described above, may also be of interest to caregivers.

In one particular embodiment, each sensing element 10 is a device, such as the one disclosed in the '142 patent, wherein the electrical resistance of each sensor element 10 changes in predictable way as each sensor element 10 is bent. In this embodiment, electrical input signal 20 that is provided to each sensor element 10 is an input voltage having a particular voltage level, and each sensor element 10 also includes or is operatively coupled to a voltage divider circuit which, for each sensing element 10, produces an output signal 22 that is an output voltage having a level that is indicative of the current resistance of the associated sensor element 10. Processor 12 may then, upon receipt of each output voltage, determine the current resistance of the associated sensor element 10 and from that resistance determine a measure of the degree of curvature of the sensing element 10. As described elsewhere herein, each measure of curvature determined in this manner may then be used to determine a representation of the curvature and/or outer contour/shape of a loop formed by sensor strip 4, such as when sensor strip 4 is looped around the head of an infant.

Sensor strip 4 (including associated circuitry) may, in one particular embodiment, for safety and environmental protection be enclosed in a sheath, protected between laminated layers of material, or molded into a compliant structure.

According to an aspect of the present invention, measuring apparatus 2 as described herein may be integrated into existing caregiver workflows (which currently use measuring tapes to measure infant head circumference) and used to compute, store and track the curvature of an infant's head over time to indicate trends to caregivers and allow early intervention. In one particular embodiment, measuring apparatus 2 may be used to periodically determine and store representations of the curvature of the infants head. Those representations may then downloaded to a computer, such as a PC, of the caregiver (by coupling electronic system 6 to the computer through an appropriate connection such as using a USB cable or a docking station coupled to the computer) so that they can be stored, displayed and analyzed over time. For example, the measured curvature information may be displayed graphically or numerically, or an overall index of curvature severity may be determined and displayed. This index would provide a quick indication of how “out-of-round” the loop measured actually is. In a perfectly round loop, all sensor elements 10 would measure the exact same radius of curvature. In an elongated curved closed loop, some sensor elements 10 would indicate a smaller radius of curvature than other sensor elements 10. An index of curvature deformity could be determined by calculating a ratio (referred to as a curvature deformity index ratio) comparing the smallest indicated radius of curvature from a sensor element or elements 10 to the largest indicated radius of curvature from a sensor element or elements 10. If a set of sensor elements 10 is considered, the curvature deformity index ratio could be further modified or weighted based on the number of sensor elements 10 meeting the “low” or “high” threshold to determine whether the deformity is in the general shape, or simply a localized anomaly.

In another particular embodiment, the representations may be uploaded to a computer network, such as a hospital computer network, for storage and/or analysis either through the computer described above or directly from measuring device 2. Alternatively, those representations may be accessed from electronic system 6 (using user interface 16) manually and recorded in the medical record of the infant for later analysis. Also, measuring device 2 can collect and stored representations for multiple infants and download and/or upload those representations as described. For example, a nurse could use measuring device 2 to collect data for a number of infants in a nursery and then download and/or upload that for storage in association with each infant's medical record.

In addition, physicians responsible for the care of infants could use measuring apparatus 2 to monitor the progress of a particular therapy or treatment for plagiocephaly (such as a therapy or treatment that employs an orthotic device), or provide early detection of positional plagiocephaly during routine office visits. Other potential areas of application of measuring apparatus 2 include reconstructive plastic surgery, and similar applications where measurement of curvature of a body part is important to providing appropriate care.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. An apparatus for measuring a characteristic of a body part, comprising:
an elongated, flexible, inextensible substrate;
a plurality of sensing elements provided along the substrate, each of the sensing elements (i) having a component wherein an electrical characteristic of the component changes predictably in response to the component being bent, and (ii) providing a signal that is indicative of a current value of the electrical characteristic; and
an electronic system, each of the sensing elements being operatively coupled to the electronic system, the electronic system being structured to receive the signal of each of the sensing elements and determine a measure of a degree of curvature of the sensing element based on the received signal associated with the sensing element.

2. The apparatus according to claim 1, wherein the electronic system is also structured to determine, based on the measure associated with each of a selected one or more of the sensing elements, a representation of a curvature of a segment defined by the selected one or more of the sensing elements.

3. The apparatus according to claim 2, wherein the segment is a closed loop.

4. The apparatus according to claim 3, wherein the electronic system is also structured to determine that the closed loop has been formed, and perform a calibration adjustment for each of the selected one or more of the sensing elements.

5. The apparatus according to claim 3, further comprising a mechanism for determining that the closed loop has been formed and for identifying the selected one or more of the sensing elements.

6. The apparatus according to claim 5, wherein the mechanism includes a plurality of conductive pads provided along the substrate and a conductive element provided at a distal end of the substrate.

7. The apparatus according to claim 2, further comprising a mechanism for identifying the sensing elements making up the selected one or more of the sensing elements.

8. The apparatus according to claim 7, wherein the mechanism includes a plurality of conductive pads provided along the substrate and a conductive element that is slideable along the substrate.

9. The apparatus according to claim 3, wherein the selected one or more of the sensing elements are less than all of the plurality of sensing elements.

10. The apparatus according to claim 1, wherein the measure of a degree of curvature of the sensing element is a radius of curvature.

11. The apparatus according to claim 1, wherein the electronic system is also structured to determine an index of curvature deformity based on the measure associated with each of a selected set of the sensing elements, the index of curvature deformity being determined by calculating a ratio comparing a smallest one of the measures associated with each of the selected set of the sensing elements to a largest one of the measures associated with each of the selected set of the sensing elements.

12. The apparatus according to claim 11, wherein the index of curvature deformity is weighted based on a number of sensor elements having the smallest one of the measures and a number of the sensor elements having the largest one of the measures.

13. The apparatus according to claim 2, wherein the representation is a radius of curvature.

14. The apparatus according to claim 2, wherein the electronic system is also structured to determine curvilinear distance of the segment.

15. The apparatus according to claim 1, wherein the electrical characteristic is one of resistance, inductance and capacitance.

16. The apparatus according to claim 1, wherein the substrate, the sensing elements and the electronic system, are provided together in a portable device.

17. A method of measuring a characteristic of a body part, comprising:
providing a plurality of sensing elements, each of the sensing elements (i) having a component wherein an electrical characteristic of the component changes predictably in response to the component being bent, and (ii) providing a signal that is indicative of a current value of the electrical characteristic;
wrapping the sensing elements around at least a portion of the body part;
receiving the signal of each of the sensing elements when the sensing elements are wrapped around the at least a portion of the body part; and
determining a measure of a degree of curvature of each of the sensing elements based on the received signals.

18. The method according to claim 17, further comprising determining based on each measure a representation of a curvature of a segment defined by the plurality of the sensing elements when the sensing elements are wrapped around the at least a portion of the body part.

19. The method according to claim 17, wherein the body part is a head.

20. The method according to claim 18, wherein the segment is a closed loop.

21. The method according to claim 20, further comprising determining that the closed loop has been formed, and performing a calibration adjustment for each of the plurality of the sensing elements.

22. The method according to claim 17, further comprising determining an index of curvature deformity based on the measure associated with each of the sensing elements, the index of curvature deformity being determined by calculating a ratio comparing a smallest one of the measures to a largest one of the measures.

23. The method according to claim 22, wherein the index of curvature deformity is weighted based on a number of sensor elements having the smallest one of the measures and a number of the sensor elements having the largest one of the measures.

24. The method according to claim 17, wherein the measure of a degree of curvature of each of the sensing elements is a radius of curvature.

25. The apparatus according to claim 18, wherein the representation is a radius of curvature.

26. The method according to claim 13, further comprising determining the curvilinear distance of the segment.

27. The method according to claim 17, wherein the electrical characteristic is one of resistance, inductance and capacitance.

28. The method according to claim 18, further comprising storing the representation and subsequently downloading the representation to a computing device.

29. The method according to claim 18, further comprising performing the wrapping, receiving and determining steps a number of additional times to generate a number of additional curvature representations, and storing and tracking over time the representation of a curvature and the plurality of additional curvature representations.
